(19) United States
(12) Patent Application Publication
(10) Pub. No.: US 2001/0052138 A1
(43) Pub. Date: Dec. 13, 2001

(54) Pepper plants which contain a single, dominant gene and which are resistant to cucumber mosaic virus

(75) Inventor: John Kao, Davis, CA (US)

Correspondence Address: ROCKEY, MILNAMOW & KATZ, LTD., TWO PRUDENTIAL PLAZA, STE. 4700, 180 NORTH STETSON AVENUE, CHICAGO, IL, 60601, US

(73) Assignee: Seminis Vegetable Seeds, Inc.

(21) Appl. No.: 09/842,475

(22) Filed: Apr. 25, 2001

Related U.S. Application Data
(60) Provisional application No. 60/202,258, filed on May 5, 2000.

(51) Int. Cl. A01H 5/00

(52) U.S. Cl. 800/279; 800/317.1

(57) ABSTRACT

The present invention relates to a pepper cultivar containing a single dominant gene which when expressed in said cultivar confers complete resistance to cucumber mosaic virus to said cultivar. The present invention also relates to methods for making a completely cucumber mosaic virus resistant pepper cultivar.

### RELATED APPLICATION INFORMATION

This application claims priority from U.S. application Ser. No. 60/202,258 filed on May 5, 2000.

### TECHNICAL FIELD

The present invention relates to pepper plants which contain a single, dominant gene in their genome, which when expressed, confers complete resistance to cucumber mosaic virus to said plants. The present invention further relates to methods of breeding said cucumber mosaic virus resistance into pepper plants.

### BACKGROUND OF THE INVENTION

The cultivated pepper, Capsicum annuum, is an important vegetable crop worldwide. There are many varieties of Capsicum annuum, such as, for example, bell pepper, sweet pepper, green pepper, red pepper and hot peppers. Varieties or cultivars of hot peppers include, for example, Anaheim, Ancho, Cascabel, Cayenne, Charleston hot, Cherry, Chilaca, Chipotle, Fresno, Gaujillo, Habanera, Jalapeno, Pasilla and Pepperoncini.

Cucumber mosaic virus (hereinafter “CMV”) is a member of the cucumovirus group. CMV is polyhedral with single-stranded (+) RNA packaged in protein subunits. RNAs 1 and 2 of CMV are associated with the replication of the viral genome, whereas RNA 3 contains both the viral coat protein gene and the 3a gene, which encodes a protein thought to be involved in the cell-to-cell movement of CMV. Owen, J., et al., J. Gen. Virol., 71:2243-2249 (1990). In addition, there is a fourth RNA, RNA 4, which is subgenomic to RNA 3 and is reported to serve as a monocistronic messenger for in vitro synthesis of the coat protein. Hayakawa, T., et al., J. Gen. Virol., 70:499-504 (1989). Some strains of CMV have a small virus-dependent satellite RNA, RNA 5, which has been demonstrated to alter the symptoms of disease. Id.

CMV is one of the most widespread and economically important plant viruses due to its wide host range and the large number of different strains isolated. The virus has a world-wide distribution and infects a variety of plants, such as tobacco, tomato, cucumber and pepper, and acts as an etiologic agent. Additionally, more than sixty (60) strains have been reported and identified and they appear to fall into two biologically and biochemically distinguishable groups—I and II. Subgroup I includes CMV strains C, D, Fny, and Y. Subgroup II includes CMV strains Q and WL.

CMV is primarily transmitted by many species of aphid in a non-persistent manner although mechanical transmission through sap carried on the hands of workers can also occur. In many plants, such as pepper, the control of CMV by chemical spray has been ineffective. While there is one pepper variety known as “Rama” which has been described in the literature by INRA, as containing “dominant CMV resistance”, it has in fact been determined that this variety is not completely resistant to infection by CMV. Instead, it has been determined that when “Rama” is exposed to certain strains of CMV that necrotic spots develop. Such necrotic spots are indicative of CMV infection.

Genetic engineering has also been used to confer resistance to CMV. Specifically, coat protein (hereinafter “CP”) mediated protection has been used to create transgenic plants which demonstrate a significant degree of protection against CMV when challenged with CMV strains from either subgroup. For example, Quemada, H., et al., Phytopathology, 81(7):794-802 (1991) engineered the coat protein gene from CMV strain C for expression in plants. Specifically, Quemada et al. transferred the coat protein gene from the C strain of CMV into the genome of tobacco. Transgenic tobacco plants containing this coat protein gene were infected with CMV strains C and Chi of subgroup I and strain WL of subgroup II, either mechanically or by aphids. Quemada et al. found that the effectiveness of the protection varied in different transgenic plant lines, ranging from almost complete to none, depending upon the challenged strain.

While genetic engineering has proven to be useful to confer resistance to CMV in various plants, genetically engineered plants are subject to a number of regulatory procedures and practices in the United States by the U.S. Department of Agriculture, the Food and Drug Administration and the Environmental Protection Agency. Because of these regulatory procedures and practices, it often takes a great deal of time and expense to bring a genetically engineered plant product to market. Therefore, there is a need in the art for new non-transgenic plant varieties developed through plant breeding, which contain genes which when expressed in a plant variety, confer resistance to CMV.

### SUMMARY OF THE PRESENT INVENTION

The present invention relates to a pepper cultivar containing a single dominant gene within its genome, which when expressed in said cultivar, confers complete resistance to cucumber mosaic virus to said cultivar.

The present invention also relates to a method for developing a pepper cultivar which is completely resistant to cucumber mosaic virus. The first step of the method involves crossing a first pepper cultivar which contains a single dominant gene within its genome, which when expressed in said cultivar confers complete resistance to cucumber mosaic virus to said cultivar with a second pepper cultivar which does not contain a single dominant gene in its genome which upon expression, encodes for complete resistance to cucumber mosaic virus. The next step involves performing one or more selection and crossing steps until a pepper cultivar is produced which contains a single dominant gene within its genome, which when expressed in said pepper cultivar, confers complete resistance to cucumber mosaic virus. The present invention also relates to a pepper cultivar produced by this method and to pepper seeds obtained from said pepper cultivar.

The pepper cultivar of the present invention can be a bell pepper, sweet pepper, green pepper, red pepper or hot pepper.

The present invention also relates to a DNA marker linked to a single dominant gene, which when said single dominant gene is expressed in a pepper cultivar, confers complete resistance to cucumber mosaic virus, wherein said DNA marker is identified using bulk segregant analysis.

Finally, the present invention relates to a method for identifying a single dominant gene in a genome of a plant, which when expressed in said plant, confers complete resistance to cucumber mosaic virus to said plant. The first step of the method involves screening the genome of a plant with the hereinbefore described DNA marker. Based upon this screening, the next step involves identifying a single dominant gene within the genome of the plant, which when expressed in said plant, confers complete resistance to cucumber mosaic virus.

### DETAILED DESCRIPTION OF THE PRESENT INVENTION

As embodied and described herein, the present invention is directed to a pepper (Capsicum) cultivar which is completely resistant to CMV. More specifically, the pepper cultivar of the present invention contains a single dominant gene within its genome. When this single dominant gene is expressed in said cultivar, it confers complete resistance to CMV to said cultivar. Additionally, the present invention relates to methods of making a completely CMV-resistant pepper cultivar.

The pepper cultivar of the present invention is stable, as evidenced by the stability of this single dominant gene through sexual crosses. Depending upon the cultivar; however, the plant size and the fruit size may be affected by environmental factors without any variance in a cultivar's resistance to CMV.

The methods of the present invention enable the preparation of pepper cultivars which display a dominant and complete resistance to CMV. The resistance manifests itself in a complete absence of symptoms and an evident incapability of virus spreading from the site of inoculation to the other parts of the plant. The following disease rating scale can be used to evaluate whether pepper cultivars developed by the methods described herein are resistant to CMV. The disease rating scale is a visual disease score based on a numerical designation from 1 to 5. This disease rating scale is more specifically described as follows: 1 indicates no symptoms of CMV; 2 indicates no symptoms of CMV; however, for some reason unrelated to CMV infection, the plant does not look as healthy as a plant rated as 1, for example, the plant may be small in size, have distorted leaves, etc.; 3 is based on the symptoms of mottling, stunting and chlorosis; 4 is based on the symptoms of severe mottling, stunting or chlorosis; 5 is a plant that is dead. Pepper cultivars with a disease rating of 1 or 2 are considered to be completely resistant to CMV, while pepper cultivars with a disease rating of 3 or greater are considered susceptible to CMV.

The methods described herein can be used to create any type of pepper cultivar having dominant and complete resistance to CMV. For example, the method described herein can be used to create dominant and completely resistant bell peppers, sweet peppers, green peppers, red peppers and/or hot peppers (such as, Anaheim, Ancho, Cascabel, Cayenne, Charleston hot, Cherry, Chilaca, Chipotle, Fresno, Gaujillo, Habanera, Jalapeno, Pasilla and Pepperoncini).

Method of Crosses

Because pepper flowers are self-pollinating, flowers to the plant to be used as a female parent in a cross are typically emasculated to prevent self-pollination. Emasculation typically involves anther removal prior to pollination. The stigmatic surface is receptive for pollen following emasculation. Flowers to be used as the source of male parent pollen may be picked from the plant and used to pollinate from 3 to 5 flowers of the same cross combination. Pollen from the male parent is then applied to the stigmatic surface of the female parent. Accordingly, an abundance of pollen may be delivered to the stigmatic surface. Preferably, each pollinated flower is marked to identify the date of pollination and the male and female parents.

Ripening of the fruit generally occurs at about 10 weeks after pollination, depending upon the environmental conditions. The collected seeds are typically cleaned by hand, and the seed separated from the fruit and stored in paper bags. Cool and cloudy weather increases the time required for the ripening of the pepper fruit.

Flowers from peppers maintain functional male and female organs. Thereupon, incorporation of the single dominant gene, which upon expression in said pepper confers complete resistance to CMV, into other pepper cultivars is possible. Therefore, this single dominant gene can be incorporated into multiple cultivars of pepper with different genetic backgrounds and combined with a wide range of known and desirable characteristics.

Strategy of Crossing

The completely CMV resistant pepper cultivars of the present invention can be produced using known breeding techniques. A breeding program can be undertaken, such as the those shown in the examples contained herein, using a pepper cultivar which contains a single dominant gene in its genome, which when expressed in said cultivar, confers complete resistance to CMV infection. Such a cultivar can be used as either the male or female parental strain in order to develop new pepper cultivars which are completely resistant to CMV. The cayenne pepper cultivar “Wonder Hot”, commercially available from Hungnong Seed America, Inc., 3065 Pacheco Pass Highway, Gilroy, Calif. 95020, can be used in the method of the present invention. “Wonder Hot” contains a single a single dominant gene, which upon expression, encodes for complete CMV resistance. “Wonder Hot” also has a strong stem with dark green leaves which allows it to grow well under unsuitable growing conditions. Additionally, it produces long straight and smooth fruits.

Breeding of a completely CMV resistant pepper is initiated by crossing a first pepper breeding line containing commercially desirable characteristics with a second pepper breeding line containing a single dominant gene in its genome which encodes for complete CMV resistance, such as the hybrid line “Wonder Hot”. To use “Wonder Hot” for breeding purposes, it must be self-pollinated several times in order to fix the single dominant gene which upon expression encodes for complete CMV resistance. The first and second pepper breeding lines may both be the same type of pepper, such as a bell or sweet pepper, or may be different types of pepper such as a sweet pepper and a Jalapeno pepper. Progeny containing the single dominant gene which upon expression encodes for complete CMV resistance are selected and maintained in the breeding program. In order to obtain a pepper cultivar which contains the single dominant gene which encodes for complete CMV resistance and which has commercially desirable characteristics, additionally crossings and selections are conducted until a commercially desirable, completely CMV resistant pepper cultivar is obtained.

For example, a first pepper cultivar containing a single dominant gene within its genome which encodes for complete CMV resistance can be crossed with a second pepper cultivar which does not contain a single dominant gene in its genome which upon expression, encodes for complete resistance to cucumber mosaic virus. The seeds from this cross are then collected and planted. Resulting plants containing the single dominant gene in its genome which upon expression, encodes for complete resistance to cucumber mosaic virus and which also possess commercially desirable characteristics, are selected and allowed to self-pollinate. The seeds resulting from this cross are then collected and planted. The resulting plants are tested for CMV resistance and the resistant plants backcrossed with a recurrent parent, which is the second pepper cultivar used in the initial cross and which does not contain a single dominant gene in its genome, which upon expression, encodes for complete resistance to cucumber mosaic virus. The seeds resulting from this cross are collected and planted and then the resulting plants containing the single dominant gene in its genome which upon expression, encodes for complete resistance to cucumber mosaic virus and which also possess commercially desirable characteristics are selected and allowed to self-pollinate. These steps of self-pollination and backcrossing are allowed to continue for a sufficient number of generations until plants are obtained which contain the single dominant gene in its genome, which upon expression, encodes for complete resistance to cucumber mosaic virus, in a homozygous condition. Once such a plant is obtained, it can then be crossed with another plant having commercially desirable characteristics in a homozygous condition in order to produce a commercially desirable hybrid plant containing a single dominant gene in its genome which upon expression, encodes for complete resistance to cucumber mosaic virus.

Alternatively, a first pepper cultivar containing a single dominant gene within its genome which encodes for complete CMV resistance can be crossed with a second pepper cultivar which does not contain a single dominant gene in its genome which upon expression, encodes for complete resistance to cucumber mosaic virus. The seeds from the cross are then collected and planted. Resulting plants containing the single dominant gene in its genome, which upon expression, encodes for complete resistance to cucumber mosaic virus, and which also possess commercially desirable characteristics, are tested for CMV resistance and the resistant plants are selected and immediately backcrossed with the recurrent parent described previously. The seeds from the cross are then collected and planted. Resulting plants containing the single dominant gene in its genome, which upon expression encodes for complete resistance to cucumber mosaic virus, and which also possess commercially desirable characteristics, are selected for CMV resistance and immediately backcrossed with the recurrent parent. The seeds from this cross are collected and planted. This process of selection and repeated backcrossing is allowed to continue for a sufficient number of generation until plants are obtained which contain a single dominant gene in its genome, which upon expression encodes for complete resistance to cucumber mosaic virus, in a homozygous condition. Once such a plant is obtained, it is allowed to self-pollinate in order to stabilize the dominant gene in the homozygous condition. Once such a plant is obtained, it can then be crossed with another plant having commercially desirable characteristics in a homozygous condition in order to produce a commercially desirable hybrid plant.

Molecular Characterization of the Single Dominant Gene

Molecular techniques, which are well known in the art, can be used to identify, locate and isolate the single dominant gene described herein, as well as markers linked to said gene. For example, bulk segregant analysis (hereinafter “BSA”) can be used to identify markers which are linked to the single dominant gene described herein (see Michelmore, R. W., et al., Proc. Natl. Acad. Sci. USA, 88:9828-9832 (1991)). BSA is effective for high resolution mapping to find genes controlling simple inherited traits, such as disease resistance genes. Indirectly, BSA can be used to saturate linkage maps and to screen for markers linked to quantative trait loci (hereinafter “QTL”).

In BSA, DNA samples from different individual progeny in a family are pooled into bulked samples by genotypic or by phenotypic class (Liu, B., Statistical Genomics: Linkage, Mapping, and QTL Analysis, CRC Press, 1998). The success of bulking by phenotypes is dependent on the correspondence of genotype and phenotype. Id. A specific target allele will occur in one bulked sample, but not the other. This pattern of frequency difference will also be seen for any marker or gene that is tightly linked to the target allele. Id. A polymorphic marker which shows a clear difference between the two bulks is likely to be linked to the target genes or nearby markers. Id. The two bulks show no differences for the rest of the genome. Id.

PCR based markers are commonly used in BSA. For RAPD markers, the principle behind BSA is that the low frequency allele will not be amplified. Id. The minimum frequency of an allele in a DNA sample that will allow the allele to be amplified in the PCR reaction is defined as sensitivity. A detailed discussion of this methodology is provided in Michelmore, R. W., et al., Proc. Natl. Acad. Sci. USA, 88:9828-9832 (1991).

Once a marker which is linked to the single dominant gene described herein is identified, this marker can be used to identify the presence of this single dominant gene in other germplasm, such as, but not limited to, tomato, melons, etc. Once such a single dominant gene is identified in other germplasm, it can be bred into lines having other commercially desirable traits using the techniques described herein.

By way of example, and not of limitation, examples of the present invention shall now be given.

### EXAMPLE 1

The pepper cultivars listed below in Table 1 were screened for CMV resistance. Ten seeds of each of the pepper cultivars listed below in Table 1 were planted in a flat, incubated in a greenhouse until the seedlings reached the cotyledon-stage (fully expanded cotyledons). Each cotyledon was mechanically inoculated with CMV strain 144-I at a concentration of 1:50 (1 gram of fresh infected grey zucchini leaf tissues in 50 ml of phosphate buffer, p.H. 7.0). CMV strain 144-I is a pepper isolate and is available from Professor Bryce Falk at the University of California at Davis, Davis, Calif.

Yolo wonder B (hereinafter “Yolo B”) is an open-pollinated pepper variety produced by Seminis Vegetable Seeds, Inc., the assignee of the present invention. S-20-1 is a selection from the variety Perennial, which was first reported by Pochard of INRA, France .

WonderHot F₅ was made by five (5) consecutive selfings of hybrid WonderHot and field selection for phenotypes such as vigor, type and absence of disease symptoms at Seminis Thailand Station in Thailand in Chin rai.

Three weeks after inoculation, symptoms were scored using the following disease rating scale: 1 indicates no symptoms of CMV; 2 indicates no symptoms of CMV; however, for some reason unrelated to CMV infection, the plant does not look as healthy as a plant rated as 1, for example, the plant may be small in size, have distorted leaves, etc.; 3 is based on the symptoms of mottling, stunting and chlorosis; 4 is based on the symptoms of severe mottling, stunting or chlorosis; 5 is a plant that is dead. Pepper cultivars with a disease rating of 1 or 2 are considered resistant to CMV, while pepper cultivars with a disease rating of 3 or greater are considered susceptible to CMV.

As shown in Table 1, pepper lines 3717-1, 3175-2 and 3780-2 were each found to be one hundred percent (100%) resistant to CMV strain 144-I. The susceptibility check, Yolo B was 100% susceptible and S-20-1 plants had a disease rating from 2-4.

TABLE 1

| | No. of Plants | CMV Rating Scale | | | | | Average CMV Disease |
|---|---|---|---|---|---|---|---|
| Variety | Tested | 1 | 2 | 3 | 4 | 5 | Rating |
| 3171-1* | 9 | 5 | 4 | | | | 1.4 |
| 3171-2* | 9 | 5 | 1 | 1 | 1 | | 2.0 |
| 3171-3* | 9 | 3 | 5 | | 1 | | 1.9 |
| 3171-4* | 10 | 4 | 3 | | 3 | | 2.2 |
| 3174-1* | 9 | | | | 9 | | 4.0 |
| 3174-2* | 7 | | 5 | | 2 | | 2.6 |
| 3174-3* | 7 | | | | 7 | | 4.0 |
| 3174-4* | 7 | | | | 7 | | 4.0 |
| 3175-1* | 8 | | 5 | 1 | 2 | | 2.6 |
| 3175-2* | 6 | | 6 | | | | 2.0 |
| 3175-3* | 6 | | 1 | 5 | | | 2.9 |
| 3780-1* | 8 | 5 | | | 3 | | 2.0 |
| 3780-2* | 7 | 5 | 2 | | | | 1.2 |
| 3780-3* | 9 | 6 | 1 | | 2 | | 1.8 |
| 3780-4* | 9 | 7 | 1 | | 1 | | 1.4 |
| Yolo B | 85 | | | | 85 | | 4.0 |
| S-20-1 | 78 | | 11 | 54 | 13 | | 3.0 |

*Wonder Hot F₅

### EXAMPLE 2

Seeds of each of the pepper cultivars listed below in Table 2 were planted in a flat, incubated in a greenhouse until the seedlings reached the cotyledon-stage (fully expanded cotyledons). Each cotyledon was mechanically inoculated with a CMV strain at a concentration of 1:50 (1 gram of fresh infected grey zucchini leaf tissues in 50 ml of phosphate buffer, p.H. 7.0). The CMV strains used in this screening were CMV strain fern leaf (an isolate from Italy), CMV strain Woodland (a proprietary CMV strain owned by Seminis Vegetable Seeds, Inc., the assignee of the present invention), CMV strain C and CMV strain 144-I. Each of the pepper cultivars was screened for CMV resistance using the procedures and rating scale described in Example 1 and the results are shown below in Table 2.

TABLE 2

| | No. of Plants | CMV Rating Scale | | | | |
|---|---|---|---|---|---|---|
| Variety | Tested | 1 | 2 | 3 | 4 | 5 |
| CMV-fern leaf | | | | | | |
| Yolo B | 11 | | | 11 | | |
| S-20-1 | 12 | | 12 | | | |
| 3171-1 | 3 | 3 | | | | |
| 3175-2 | 12 | 12 | | | | |
| CMV-Woodland | | | | | | |
| Yolo B | 11 | | | | 9 | 2 |
| S-20-1 | 12 | | 11 | | 1 | |
| 3171-1 | 9 | 9 | | | | |
| 3175-2 | 11 | 9 | 2 | | | |
| CMV-C | | | | | | |
| Yolo B | 10 | | | | 7 | 3 |
| S-20-1 | 12 | | | 12 | | |
| 3171-1 | 6 | 6 | | | | |
| 3175-2 | 12 | 7 | | 4 | 1 | |
| CMV-144-I | | | | | | |
| Yolo B | 12 | | | | 2 | 10 |
| S-20-1 | 12 | | 12 | | | |
| 3171-1 | 4 | 4 | | | | |
| 3175-2 | 11 | 9 | 1 | 1 | | |

### EXAMPLE 3

Seeds of each of the pepper cultivars listed below in Table 3 were planted in a flat, incubated in a greenhouse until the seedlings reached the cotyledon-stage (fully expanded cotyledons). Each cotyledon was mechanically inoculated with CMV strain 144-I at a concentration of 1:50 (1 gram of fresh infected grey zucchini leaf tissues in 50 ml of phosphate buffer, p.H. 7.0).

The pepper lines 3171-1, 3172-2, 3171-3, 3175-1, 3175-2, 3180-1, 3180-2, 3180-3 listed below in Table 3 are all self-pollinated progeny (F₆) from the cultivars listed in Example 1. Each of these cultivars was screened for CMV resistance using the procedures and rating scale described in Example 1 and the results are shown below in Table 3.

The pepper lines 3171-1, 3171-3, 3175-1, 3175-2, 3180-1 and 3180-2 from Example 1 were each used as female parent in a cross with a proprietary sweet pepper cultivar of Seminis Vegetable Seeds, Inc., the assignee of the present invention. This proprietary sweet pepper cultivar is susceptible to CMV but is resistant to tomato spotted wilt virus (this sweet pepper cultivar is referred to herein as “TSWR”). The seeds resulting from this cross were collected and planted. The hybrids resulting from each of these crosses were then screened for CMV resistance using the procedures and rating scale described in Example 1 and the results are shown below in Table 3.

Pepper lines 3171-1, 3171-2, 3175-2 and 3180-1 from Example 1 were also used as a male parent in a cross with TSWR. The seeds resulting from this cross were collected and planted. The hybrids resulting from each of these crosses were then screened from CMV resistance using the procedures and rating scale described in Example 1 and the results are shown below in Table 3.

TABLE 3

| | No. of Plants | CMV Rating Scale | | | | |
|---|---|---|---|---|---|---|
| Lines | Tested | 1 | 2 | 3 | 4 | 5 |
| 3171-1 | 12 | 12 | | | | |
| 3171-2 | 7 | 4 | | | 3 | |
| 3171-3 | 15 | 13 | | 1 | 1 | |
| 3175-1 | 20 | 18 | 1 | 1 | | |
| 3175-2 | 19 | 14 | | | | 5 |
| 3180-1 | 8 | 7 | 1 | | | |
| 3801-2 | 19 | 15 | 1 | 3 | | |
| 3801-3 | 20 | 15 | 1 | 3 | | 1 |
| 3171-l × TSWR (F₁) | 5 | 4 | | 1 | | |
| 3171-3 × TSWR (F₁) | 4 | 1 | 1 | 2 | | |
| 3175-1 × TSWR (F₁) | 3 | 2 | 1 | | | |
| 3175-2 × TSWR | Did not germinate | | | | | |
| 3180-1 × TSWR (F₁) | 2 | | 2 | | | |
| 3180-2 × TSWR (F₁) | 5 | 5 | | | | |
| TSWR × 3171-1 (F₁) | 18 | 10 | 4 | 4 | | |
| TSWR × 3171-2 (F₁) | 18 | 7 | 1 | 3 | 4 | 3 |
| TSWR × 3175-2 (F₁) | 20 | 19 | 1 | | | |
| TSWR × 3180-1 (F₁) | 20 | 6 | 3 | 6 | 4 | 1 |
| Yolo B | 37 | | | | 4 | 33 |
| S-20-1 | 36 | | 11 | 9 | | 16 |
| TSWR | 28 | | | 2 | 12 | 14 |

### EXAMPLE 4

The individual F₁ plants resulting from the cross between TSWR×3171-1 and TSWR×3171-2, described in Example 3, were selfed and the resulting seed collected. The seed was then planted and the cotyledons of the resulting F₂ plants inoculated with CMV strain 144-I as described in Example 1. These plants were then screened for resistance to this CMV strain 144-I using the rating scale described in Example 1 and the results are shown below in Table 4.

Line 3171-1, the resistant male parent and TSWR, the susceptible female parent, were both included in the test for comparison.

TABLE 4

| | | | | | | | % Resis- |
|---|---|---|---|---|---|---|---|
| | No. of | | | | | | tance |
| | Plants | CMV Rating Scale | | | | | (No. of |
| Line | Tested | 1 | 2 | 3 | 4 | 5 | R/total) |
| TSWV × 3171-1 (F₂) | 53 | 28 | 9 | 7 | 4 | 5 | 70% |
| TSWV × 3171-2 (F₂) | 43 | 26 | 2 | 5 | 5 | 5 | 65% |
| 3171-1 R male parent | 47 | 46 | 1 | | | | 100% |
| TSMV (S female parent) | 53 | | | | 41 | 12 | 0% |
| Yolo B (Susceptible check) | 38 | | | | 17 | 21 | 0% |

The F2 segregation ratio (R:S) for both F₂ populations is closed to 3:1. This demonstrates that the gene conferring CMV resistance is: (a) a single gene; and (2) dominant.

### EXAMPLE 5

This example describes how BSA can be used to find one or more markers linked to the single dominant gene described herein, which when expressed in the genome of a plant, namely pepper, encodes for complete resistance to cucumber mosaic virus.

A bulked sample is prepared from a CMV susceptible pepper line and another bulked sample is prepared from CMV resistant pepper line containing the single dominant gene described herein. A DNA marker that is independent of the CMV resistance gene will be represented as a band in both the resistant and susceptible bulked sample gel lanes. A DNA marker linked to the dominant CMV disease resistance should be represented as a band in one of the bulked sample lanes, but not the other. However, sometimes, a band from an unlinked marker will be present in the lane for one bulked sample, but not for the other. This outcome is referred to as a “false positive.” A “false negative” outcome occurs if a DNA marker linked to the dominant CMV resistance gene produces a band in both bulked sample lanes. The control of false positives and false negatives is important in BSA. Probabilities of false positives and false negatives vary, depending on the types of mapping populations (backcross vs. F2), the bulk size and sensitivity.

The two bulk samples can then be screened for differences with several RFLP probes simultaneously or with individual RAPD primers. Linkage between a polymorphic marker and the target locus (the single dominant gene) is then confirmed and quantified by using the segregating population from which the bulk samples were generated. Probes or primers for loci that are polymorphic and absolutely linked to the single dominant gene described herein will detect clear differences between the bulk samples. Unlinked loci will appear heterozygous with approximately equal band intensities in each bulk sample. Recombination between the target marker and the assayed polymorphic locus will result in diminishing distinction between the two bulk sample with decreasing linkage until the locus appears unlinked.

All references cited herein are hereby incorporated by reference.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby.

Changes can be made to the composition, operation and arrangement of the method of the present invention described herein without departing from the concept and scope of the invention as defined in the following claims.

What is claimed is:

1. A method for developing a pepper cultivar which is completely resistant to cucumber mosaic virus, the method comprising the steps of:
a. crossing a first pepper cultivar which contains a single dominant gene within its genome, which when expressed in said cultivar confers complete resistance to cucumber mosaic virus to said cultivar with a second pepper cultivar which does not contain a single dominant gene in its genome which upon expression, encodes for complete resistance to cucumber mosaic virus;
b. performing one or more selection and crossing steps until a pepper cultivar is produced which contains a single dominant gene within its genome, which when expressed in said pepper cultivar, confers complete resistance to cucumber mosaic virus.

2. The method of claim 1 comprising two or more selection and crossing steps.

3. The method of claim 1 wherein the first pepper cultivar is used as the female parent.

4. The method of claim 1 wherein the first pepper cultivar is used as the male parent.

5. The method of claim 1 wherein the pepper cultivar is a bell pepper, sweet pepper, green pepper, red pepper or hot pepper.

6. A pepper cultivar which is completely resistant to cucumber mosaic virus produced by the method of claim 1.

7. Progeny from the pepper cultivar of claims 6.

8. Pepper seeds from the pepper cultivar of claims 6 or 7.

9. The method of claim 1 wherein the first pepper cultivar is Wonder Hot.

10. A pepper cultivar which is completely resistant to cucumber mosaic virus produced by the method of claim 9.

11. Progeny from the pepper cultivar of claim 10.

12. Pepper seeds from the pepper cultivar of claims 10 or 11.

13. A DNA marker linked to a single dominant gene, which when said single dominant gene is expressed in a pepper cultivar, confers complete resistance to cucumber mosaic virus, wherein said DNA marker is identified using bulk segregant analysis.

14. A method for identifying a single dominant gene in a genome of a plant, which when expressed in said plant, confers complete resistance to cucumber mosaic virus to said plant, the method comprising the steps of:
screening the genome of a plant with the DNA marker of claim 13 and identifying a single dominant gene within the genome of the plant which when expressed in said plant confers complete resistance to cucumber mosaic virus.
